(19) United States
(12) Patent Application Publication
(10) Pub. No.: US 2006/0194305 A1
(43) Pub. Date: Aug. 31, 2006

(54) Micro-holding pipette with an oblique opening

(76) Inventors: Jiahui Xia, Hunan (CN); Qifeng Lyu, Hunan (CN)

Correspondence Address: BIRCH STEWART KOLASCH & BIRCH, PO BOX 747, FALLS CHURCH, VA, 22040-0747, US

(21) Appl. No.: 10/565,655

(22) PCT Filed: May 19, 2003
(86) PCT No.: PCT/CN03/00363

§ 371 Date: Jan. 24, 2006

(51) Int. Cl. C12M 3/00

(52) U.S. Cl. 435/285.1

(57) ABSTRACT

The present invention relates to a micro-holding pipette. The holding end of the micro-holding pipette is a oblique opening, i.e. the end surface of the opening of the holding end of the micropipette is at an acute angle with the ordinate axis of the holding end, which is less than 90°, and preferably is from 25° to 85°. The present invention advantageously allows piercing a pellucid zona from the side, that is not the obverse surface, of a ovum or embryo by a micro injection needle or assistant needle for incubating, and without the deformation of the ovum or embryo due to the pressure. It is beneficial to various micro-processes. The novel micro-holding pipette and its operation can significantly prevent damage to the embryo and improve the healthy growth of the embryo.

### FIELD OF THE INVENTION

The present invention relates to a micro-holding pipette.

### BACKGROUND OF THE INVENTION

A micro-holding pipette is a micropipette that is used to hold or fix an ovum or embryo of a human or animals in a microscope by negative pressure. Until to now, all kinds of micro-holding pipette have blunt ends, i.e. their surfaces are at a blunt angle (90°) to the ordinate axis of the holding end (FIG. 1 and FIG. 2). A micro-holding pipette is widely used in assisted reproductive technology (ART), for example, intracytoplasmic sperm injection (ICSI), assisted hatching (AH) and preimplantation genetic diagnosis (PGD). The ovum or embryo is held at the 9 o'clock position by a micro-holding pipette and another micropipette, an ICSI needle or a hatching needle opposite to the micro-holding pipette pierces the zona pellucid (ZP) of the ovum or embryo at 3 o'clock opposition during micro-processes. However, if the ICSI needle or hatching needle pierces the ZP at other oppositions, for example 5 o'clock opposition, the ovum or embryo would be deformed or damaged due to the interlace mechanical pressure as both the holding force and pierce force are not at the same axis. So, an operator has to face much more difficulty and needs much more time in micro-processes using a traditional micro-holding pipette.

However, the ovum or embryos need to be approached at 4 o'clock to 6 o'clock positions in many micro-processes, like mechanical AH, because the ovum or embryo will not but ZP loads the mechanical pressures when the opposite micropipette pierces the ZP at 4 o'clock to 6 o'clock positions first, and then other steps are performed. The inventor has found that: although the opposite micropipette can easily pierce the ZP at 3 o'clock site, the ovum or embryo held by the traditional blunt end micro-holding pipetter would be deformed because of mechanical pressure from micropipettes.

### DESCRIPTION OF THE INVENTION

One problem solved by the present invention is that the ovum or embryo is held conveniently at 45-past-8 o'clock to 7 o'clock position and the ZP is easily pierced or drilled at the 4 o'clock to 5 o'clock position by the opposite micropipette. Characters of the present invention are that: the holding end of the micro-holding pipette is an oblique opening, i.e. the surface of the opening of the holding end of the micropipette is at an acute angle with the ordinate axis of the holding end, which is less than 90°, and preferably is from 25° to 85°.

Advantages of the present invention are that: micropipettes or needles opposite to the micro-holding pipette can pierce or drill a ZP from the side that is not the obverse surface of an ovum or embryo, and the ovum or embryo does not deform due to the pressures, and it is beneficial to various micro-processes. The novel micro-holding pipette can significantly prevent damage to the embryo and improve the healthy growth of the embryo.

### BRIEF DESCRIPTION OF THE DRAWINGS

FIG. 1 A traditional micro-holding pipette

FIG. 2 A magnification of a micro-holding pipette at the ‘A’ site

FIG. 3 The present invention

FIG. 4 A magnification of the present invention at the ‘B’ site

FIG. 5 A hatching needle piercing at 5 o'clock

FIG. 6 A hatching needle pierced at 5 o'clock

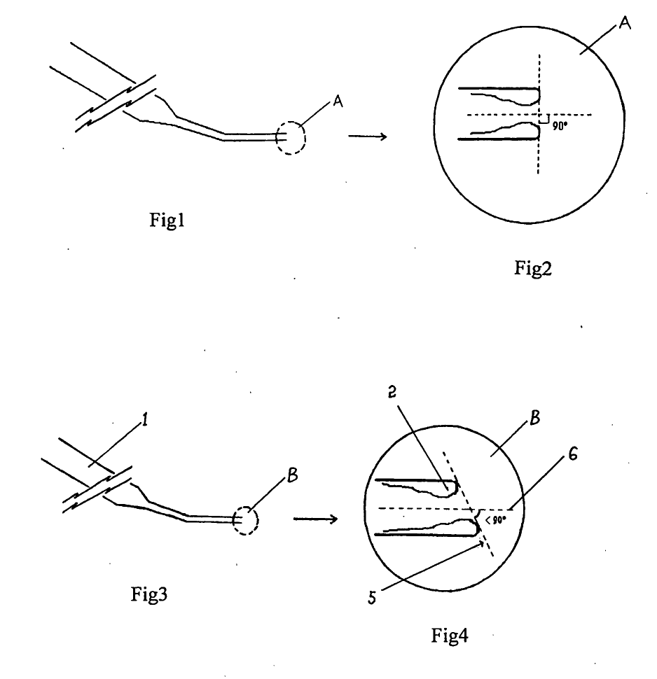

### EMBODIMENT

FIG. 3 and FIG. 4 show that the holding end of the micro-pipetter ‘1’ is an oblique opening ‘2’, i.e. the surface of the opening of the holding end ‘5’ of the micropipette is at an acute angle with the ordinate axis ‘6’ of the holding end, which is less than 90°, and preferably is from 25° to 85°.

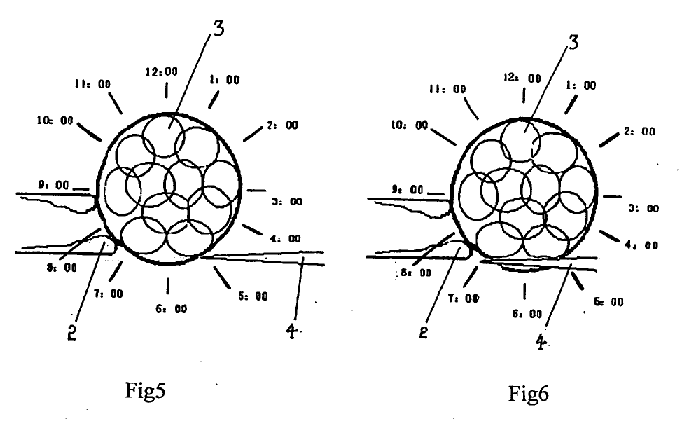

FIG. 5 shows that an embryo ‘3’ is held at the 8 o'clock position and the hatching needle ‘4’ is easily piercing a ZP ‘3’ of the embryo at the 5 o'clock position (FIG. 6).

During the manipulation, both the present invention and the hatching needle are at the same pressure axis that ensures that the needle easily pierces or drills the ZP without deformation or damaging of the embryo. But the traditional micro-pipette does not have this advantage because the embryo has to be held at the 9 o'clock position.

There are several improvements in ZP mechanical drilling with the invention: —it is more convenient and easy to pierce the ZP and less time is needed to drill the ZP in micro-processes, —there are not squeezing and buffeting to the ovum or embryo which and so mechanical harm is avoided, and —a novel method besides traditional methods can be used for ZP mechanical drilling, for example AH and PGD. The present invention is especially useful in ICSI.

1. A micro-holding pipette with an oblique opening, characterized in that the surface of the opening of the holding end of the micropipette is at an acute angle with the ordinate axis of the holding end, which is less than 90°.

2. A micro-holding pipette according to claim 1, characterized in that the acute angel preferably is from 25° to 85°.
